(19) United States
(12) Patent Application Publication
(10) Pub. No.: US 2003/0232094 A1
(43) Pub. Date: Dec. 18, 2003

(54) Composition and method for the treatment of skin irritations

(76) Inventor: Peter E. Fuller, Waukee, IA (US)

Correspondence Address: R. BURNS ISRAELSEN, WORKMAN, NYDEGGER & SEELEY, 1000 EAGLE GATE TOWER, 60 EAST SOUTH TEMPLE, SALT LAKE, UT, 84111, US

(21) Appl. No.: 10/171,253

(22) Filed: Jun. 12, 2002

(51) Int. Cl. A61K 31/60; A61K 33/00

(52) U.S. Cl. 424/717; 514/165

(57) ABSTRACT

A composition is provided that is suitable for topical administration for the treatment of a skin irritation, the composition comprising acetylsalicylic acid and sodium bicarbonate. Also provided is a method for treating a skin irritation in an individual comprising topically administering to the affected area a therapeutically effective amount of a composition comprising acetylsalicylic acid and sodium bicarbonate. The composition is particularly useful for decreasing the temperature at the surface of a sunburn and for reducing pain and discomfort caused by a sunburn.

### FIELD OF THE INVENTION

This invention relates to a composition and method for the treatment of skin irritations, such as those caused by sunburn, wounds, insect bites, and poisonous plants.

### BACKGROUND OF THE INVENTION

Although there has been substantial effort in recent years to reduce or eliminate the risk of sunburn (erythema) produced by certain wavelengths in the ultraviolet (UV) region of the spectrum, there are still circumstances wherein skin becomes exposed to UV radiation. Such exposure may, in some cases, cause sunburn that needs to be treated.

To be useful, a composition that is intended for treatment of sunburn would preferably satisfy several objectives simultaneously. The main objectives of a formulation for the treatment of sunburn are to relieve pain, eliminate the source of heat, stop the burn progression and, if necessary, help prevent infection. Thus, a useful sunburn-treating composition preferably provides immediate relief from pain while also helping to promote healing. It is also desirable that the separate components of the composition be combined in a reasonably convenient and cost-effective process and that the composition, thus prepared, remains stable during storage. Finally, it is preferable that the sunburn-treating composition be contained in a carrier container so that the formulation may be conveniently delivered and applied when needed. In addition, it is desirable for health reasons, in some cases, to package the sunburn-treating composition in single-dose packaging so as to reduce the risk of contamination from one usage to the next. After application, it is also preferable that the sunburn-treating composition provide the relief and healing effects sought without producing an uncomfortable sticky sensation and without soiling or sticking to one's clothing. The sunburn-treating composition also preferably does not produce a residue that has to be subsequently washed or removed from the sensitive burned area.

Known compositions for treating sunburn have certain disadvantages. For example, application of petroleum-based compositions to the sunburned area does not produce a sufficiently large heat transference effect to remove heat from the sunburned area. Furthermore, petroleum-based compositions tend to produce a residue that needs to be subsequently cleansed from the tender and sensitive area of sunburned skin. Such cleansing tends to cause still further discomfort.

### SUMMARY OF THE INVENTION

The inventor has discovered a novel composition that is useful for treating a variety of skin irritations, such as those caused by burns, wounds, insect bites, and poisonous plants.

In one aspect of the invention, there is provided a composition for the treatment of a skin irritation, the composition comprising acetylsalicylic acid and sodium bicarbonate.

In another aspect of the invention, there is provided a method for treating a skin irritation in an individual comprising topically administering to the affected area a therapeutically effective amount of a composition containing acetylsalicylic acid and sodium bicarbonate.

### DETAILED DESCRIPTION OF THE PREFERRED EMBODIMENT

The methods by which the objects, features and advantages of the present invention are achieved will now be described in more detail. These particulars provide a more precise description of the invention for the purpose of enabling one of ordinary skill in the art to practice the invention, but without limiting the invention to the specific embodiments described.

As used herein, the term “skin irritation” is intended to refer to any condition of the skin causing discomfort, including that caused by burns, such as sunburn, wounds, such as a laceration, insect bites, poisonous plants, and/or allergens.

As used herein, the term “therapeutically effective amount” is intended to refer to that amount necessary to bring relief to an affected tissue(s), organ(s), or organ system(s) of an individual, or to an entire individual.

As used herein, the term “topically administering” is intended to refer to the direct laying on or spreading of, a composition on epidermal tissue at the affected site of the epidermal tissue.

In one aspect of the invention, there is provided a composition for the treatment of a skin irritation, the composition comprising acetylsalicylic acid and sodium bicarbonate.

In another aspect of the invention, there is provided a method for treating a skin irritation in an individual comprising topically administering to the affected area a therapeutically effective amount of a composition containing acetylsalicylic acid and sodium bicarbonate.

The composition of the invention can exist in a variety of delivery forms, including, for example, solutions, creams, lotions, emulsions, ointments, aerosols, gels, and solids. Preferably, the composition exists in solution form, wherein the solvent is preferably water. This aqueous composition is preferably of a character that is adapted to be sufficiently thin to allow spray application but not so thin as to flow away from the treated area upon such application. It is preferable to use sterile, deionized water as the solvent, although a less grade of purified water is sufficient for the functional purposes of the invention.

Acetylsalicylic acid is preferably present in the range from about 0.1 to about 10 weight percent based on the final total weight of the aqueous composition after all components have been included in the composition. The weight percent of each component included in the aqueous composition disclosed herein is based on the final total weight of the aqueous composition. Most preferably, the acetylsalicylic acid is present in an amount corresponding to about 2.5 weight percent.

Sodium bicarbonate is preferably present in the range from about 0.1 to about 10 weight percent based on the final total weight of the aqueous composition after all components have been included in the composition. Most preferably, the sodium bicarbonate is present in an amount corresponding to about 2.5 weight percent.

In addition to acetylsalicylic acid and sodium bicarbonate, the composition of the invention can further comprise one or more of the members selected from the group consisting of acidifying agents, alkalizing agents, aerosol propellants, antimicrobial agents, antioxidants, buffering agents, chelating agents, coloring additives, dispersing agents, emollients, emulsifying agents, humectants, fragrances, masking agents, preservatives, sugars, sunscreen agents, surfactants, suspending agents, and thickening agents.

Acidifying and alkalizing agents can be added to obtain the desired pH of the composition. Examples of acidifying agents included acetic acid, citric acid, glacial acetic acid, malic acid, and proprionic acid. Examples of alkalizing agent include edetol, potassium carbonate, potassium hydroxide, sodium borate, sodium carbonate, and sodium hydroxide.

Aerosol propellants can be used when the composition is to be administered as an aerosol (solution) under pressure. Examples of aerosol propellants include halogenated hydrocarbons such as dichlorodifluoromethane, dichlorotetrafluoroethane, and trichloromonofluoromethane, nitrogen, and volatile hydrocarbons such as butane, propane, isobutane, or mixtures thereof.

Anti-microbial agents can be used when the area that the composition is to be applied is prone to microbial infection, e.g., by bacteria, fungal, or protozoa. Examples of such agents include benzyl alcohol, chlorobutanol, phenylethyl alcohol, phenylmercuric acetate, potassium sorbate, and sorbic acid, benzoic acid, butyl paraben, ethyl paraben, methyl paraben, propyl pareben, and sodium benzoate.

Antioxidants can be used to protect ingredients of the composition from oxidizing agents that are included within or come in contact with the composition. Examples of antioxidants include water-soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butytlated hydroxyanisole, butylated hydroxytoluene, potassium propyl gallate, octyl gallate, dodecyl gallate, phenyl-alpha-napthyl-amine, and tocopherols such as alpha-tocopherol.

Buffering agents can be used to maintain an established pH of the composition. Examples of buffering agents included sodium citrate, calcium acetate, potassium metaphosphate, potassium phosphate monobasic, and tartaric acid.

Chelating agents can be used to maintain the ionic strength of the composition and/or bind to destructive compounds and metals that are included within or come in contact with the composition. Examples of chelating agents included dihydroxy ethyl glycine, citric acid, tartaric acid, edatate dipotassium, edetate disodium, edetic acid, and ethylenediamine tetracetic acid (EDTA) and its salts (e.g., tetrasodium EDTA).

Coloring additives can be used to add color to the composition. Examples of such coloring additives include titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 10B, and Acid Red 51.

Examples of dispersing and suspending agents include quarternium-18 hectorite, polyhydroxy stearic acid, poligeenan and silicon dioxide.

Emollients are agents that soften and smooth the skin. Examples of emollients include hydrocarbon oils and waxes (e.g., natural and synthetic waxes) such as mineral oil, petrolatum, microcrystaline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholidds, and sterols.

Emulsifying agents can be used for preparing emulsions of the present invention. Examples of emulsifying agents used for preparing water-in-oil emulsions include cyclomethicone (and) dimethicone copolyol, dimethicone copolyol, cetyl dimethicone copolyol, PEG-30 dipolyhydroxystearate, and PEG-40 sorbitan peroleate. Examples of emulsifying agents used for preparing oil-in-water emulsions of the present invention include glyceryl stearate, PEG-100 stearate, methyl gluceth sesquisterate, fatty alcohols, and alkyl phenols condensed with ethylene oxide.

Humectants are agents that promote the retention of moisture, e.g., moisturizers. Examples of humectants include sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl-1,3-propanediol, and derivatives thereof, and hyaluronic acid.

Examples of fragrances include peppermint, rose oil, rose water, aloe vera, clove oil, menthol, camphor, eucalyptus oil, and other plant extracts. Certain fragrances may require a solubilizer, e.g., PPG-5-ceteareth-20. To eliminate certain odors from compositions, masking agents may be used. An example of a masking agent includes ethylene brassylate.

Preservatives can be used to protect the composition from degradation. Examples of preservatives include liquipar oil, phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, phenol, and mixtures thereof (e.g., liquipar oil).

Examples of sugars include monosaccharides, disaccharides, and polysccharides such as glucose, xylose, fructose, reose, ribose, pentose, arabinose, allose, tallose, altrose, mannose, galactose, lactose, sucrose, erythrose, glyceraldehyde, or any combination thereof.

Sunscreen agents are agents used to block or reduce the amount of ultraviolet radiation impinging on the skin (e.g., by absorption, scattering, and reflection of the ultraviolet radiation). Examples of sunscreen agents include both organic compounds and their salts such as octyl methoxycinnamate, octyl salicylate, benzophenone-3 homosalate, octocrylate, avobenzone, and menthyl anthranilate, as well as inorganic particulate materials such as zinc oxide, silica, iron oxide, titanium dioxide, and 2-ethyl-hexyl-p-methoxycinnamate.

Surfactants are agents used to stabilize multi-component compositions, e.g., used as wetting agents, antifoam agents, emulsifiers, dispersing agents, and penetrants. Examples of surfactants include methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, and polysorbate 85, sodium lauryl sulfate, sorbitan and its derivatives.

Preferably, the aqueous composition of the invention includes a humectant, such as glycerin. The humectant tends to reduce the tackiness produced by the aqueous composition after it has been applied and has dried on the skin. The humectant is present in an amount such as to produce an aqueous composition that is capable of being substantially non-tacky after drying on the skin. Preferably, the humectant is present in the range from about 0.1 to about 20 weight percent based on the final total weight of the water-based formulation after all components have been included in the aqueous composition. Most preferably, the humectant is present in an amount corresponding to about 5 weight percent.

It is to be understood that the above components can be mixed and/or blended together using standard techniques well known in the art to achieve the inventive composition. It is further to be understood that the order of addition of each component is not important in preparing the composition.

The aqueous composition of the invention can be sealed in an appropriate carrier or container. Such a carrier or container may be a tube, a bottle, a single-dose packet or the like. Preferably, the container is a pump spray bottle. The tube or bottle can also be collapsible so as to permit convenient application of a relatively large quantity of the aqueous composition to a sunburned area.

### EXAMPLE

### Use of Aqueous Composition to Treat Sunburn

A 19-year old male and a 17-year old female, each suffering from classical erythema of the arms and face, were subjected to topical administration of an aqueous composition comprising 2.5 weight percent acetylsalicylic acid, 2.5 weight percent sodium bicarbonate, 5 weight percent glycerin, and the balance water, and were monitored for skin surface temperature changes, with the following results (temperatures measured with a Cooper laser thermometer):

| | |
|---|---|
| 19-year old male | 17-year old female |

Surface Temperatures
Prior To Administration

| | | |
|---|---|---|
| Arms | 96° F. | 94° F. |
| Face | 98° F. | 96° F. |

Surface Temperatures 30-90
Seconds Following Administration

| | | |
|---|---|---|
| Arms | 86° F. | 86° F. |
| Face | 85° F. | 85° F. |

It is believed by the inventor that the decrease in skin surface temperature is brought about by the production of carbon dioxide by the reaction of acetylsalicylic acid and sodium bicarbonate in the presence of water. Carbon dioxide is a greenhouse gas, and is therefore effective for absorbing heat. It is further believed that the net result of combining all these components in a single, specially-formulated, aqueous composition offers a unique combination of cooling properties. Furthermore, skin irritations, such as those caused by sunburn, can be treated with the inventive, aqueous composition without producing the undesirable side-effects, such as tackiness, that are frequently encountered, for example, with a petroleum-based composition. In addition, the subject aqueous composition does not produce a residue that may need to be subsequently removed from a sensitive sunburned area.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. An aqueous composition suitable for topical administration for the treatment of a skin irritation, the aqueous composition comprising:
a solvent;
acetylsalicylic acid;
sodium bicarbonate; and
a humectant.

2. The aqueous composition of claim 1 wherein the solvent is water.

3. The aqueous composition of claim 1 wherein the humectant is glycerin.

4. The aqueous composition of claim 1 wherein acetylsalicylic acid is present in about 2.5 weight percent.

5. The aqueous composition of claim 1 wherein sodium bicarbonate is present in about 2.5 weight percent.

6. The aqueous composition of claim 3, wherein glycerin is present in about 5 weight percent.

7. The aqueous composition of claim 1, further comprising one or more members of the group consisting of an acidifying agent, alkalizing agent, antimicrobial agent, antioxidant, buffering agent, chelating agent, coloring additive, dispersing agent, emollient, fragrance, masking agent, preservative, sugar, sunscreen agent, surfactant, and suspending agent.

8. A method for treating a skin irritation in an individual comprising topically administering to the affected area a therapeutically effective amount of an aqueous composition comprising:
a solvent;
acetylsalicylic acid;
sodium bicarbonate; and
a humectant.

9. The method of claim 8 wherein the solvent is water.

10. The method of claim 8 wherein the humectant is glycerin.

11. The method of claim 8 wherein acetylsalicylic acid is present in about 2.5 weight percent.

12. The method of claim 8 wherein sodium bicarbonate is present in about 2.5 weight percent.

13. The aqueous composition of claim 10, wherein glycerin is present in about 5 weight percent.

14. The method of claim 8, wherein the aqueous composition further comprises one or more members of the group consisting of an acidifying agent, alkalizing agent, antimicrobial agent, antioxidant, buffering agent, chelating agent, coloring additive, dispersing agent, emollient, fragrance, masking agent, preservative, sugar, sunscreen agent, surfactant, and suspending agent.
